(19) United States
(12) Patent Application Publication
(10) Pub. No.: US 2018/0264285 A1
(43) Pub. Date: Sep. 20, 2018

(54) Breast Cup System and Method for Hyperthermic Anaerobic Cell Apoptosis

(71) Applicant: Rodney D. Smith, North Ogden, UT (US)

(72) Inventor: Rodney D. Smith, North Ogden, UT (US)

(21) Appl. No.: 15/462,809

(22) Filed: Mar. 18, 2017

(51) Int. Cl. A61N 5/06; A61F 7/00; A61F 7/02

(52) U.S. Cl. CPC A61N 5/0625; A61F 7/007; A61F 7/02; A61N 2005/0643; A61F 2007/0054; A61F 2007/0071; A61N 2005/0626; A61F 2007/0021

(57) ABSTRACT

A breast cup system for improving breast health and anaerobic cell apoptosis includes a breast cup configured to vascularly isolate a breast from the body thereof via an onset of a tourniquet-like suction (TSO) applied to the breast. The suspension of vascular circulation in the breast enables a hyperthermic isolation therein via retaining indigenous heat or applied external heat. A microcontroller is configured to control the TSO and various thermic devices and electromagnetic radiation (EMR) applied to the breast via the breast cup to create the hyperthermic isolation sufficient to cause apoptosis of anaerobic cells therein. The system further includes a heat transfer detection of anaerobic cell apoptosis based on a ratio of an amount of heat transferred from the breast cup over a period of time through the breast relative to a heat transfer through a healthy breast tissue of a same mass over a same period of time.

### BACKGROUND AND FIELD OF INVENTION

In any tissue, heat causes vasodilation. In a breast tumor, the microvasculature is made up of an overabundance of capillary beds which are unable to dilate and therefore inhibit the rejection of heat due to its mass (density of tissue). Accordingly, blood flow through the area is more sluggish and commensurately unable to dissipate heat generated with the cell or applied externally to the area. The inability to reject heat by dilation, as normal vasculature would, also subjects the tumor to hypoxia (oxygen deficiency), anaerobic metabolism and local acidosis and even death of an anaerobic cell.

Hyperthermia is the process of raising the body temperature, either locally or globally, for medicinal purposes. Historically, hyperthermia has been recognized for its curative power in treating tumors. The first application of hyperthermia for regional cancer control dates to 1898 when Swedish gynecologist Westermark treated cervical cancer by running hot water through an intracavitary spiral tube. However, conventional methods require specialized perfusion protocol for whole-body hyperthermia leading to instances of neurological damage in association with serum hypophosphatemia. Therefore, whole body heating is problematic and requires strict adherence to timed exposure and specific temperature control and therefore unnecessary costs and complications.

### SUMMARY OF THE INVENTION

A disclosed breast cup system for anaerobic cell apoptosis includes a breast cup configured to vascularly isolate a breast from the body thereof via an onset of a tourniquet-like suction (TSO) applied to the breast to affect a substantial vascular circulation suspension in the breast to enable a hyperthermic stasis in the breast. The system also includes a microcontroller configured to control the TSO and various thermic devices and electromagnetic radiation (EMR) applied to the breast via the breast cup to create the hyperthermic isolation in the breast sufficient to cause cell death of anaerobic cells therein. The system includes a heat transfer detector configured to determine the temperature threshold of anaerobic cell and respiratory limits based on a cooing rate of heat transferred from the breast to the breast cup and to the body over a period of time relative to a cooling rate of healthy breast tissue of a similar mass over a similar period of time. The system yet includes a heat transfer detection of anaerobic cell apoptosis based on a ratio of an amount of heat transferred from the breast cup over a period of time through the breast relative to a heat transfer through a healthy breast tissue of a same mass over a same period of time. The generation of indigenous or latent heat within diseased cells via an ATP (adenosine triphosphate) response to electromagnetic radiation is also applied to the breast via the disclosed breast cup.

A method is also disclosed for anaerobic cell death via a breast cup system. The method includes vascularly isolating a breast from the body thereof through an onset of a tourniquet-like suction (TSO) applied to the breast via a breast cup system. The TSO substantially suspends vascular circulation in the breast to enable a hyperthermic stasis in the breast. The disclosed method also includes controlling the TSO and various thermic devices and electromagnetic radiation (EMR) applied to the breast via the breast cup and a microcontroller configured to create the hyperthermic stasis in the breast sufficient to cause apoptosis of anaerobic cells therein. The method further includes determining the anaerobic cell death as a result of the TSO and the hyperthermic stasis based on one of an intrusive and a non-intrusive measurement including a ratio of an amount of heat transferred from the breast cup over a period of time through the breast relative to a heat transfer through a healthy breast tissue of a same mass over a same period of time.

Other aspects and advantages of embodiments of the disclosure will become apparent from the following detailed description, taken in conjunction with the accompanying drawings, illustrated by way of example of the principles of the disclosure.

### BRIEF DESCRIPTION OF THE DRAWINGS

FIG. 1 is a component block diagram of a breast cup system for hyperthermic anaerobic cell apoptosis in accordance with an embodiment of the present disclosure.

FIG. 2 is a block diagram of a method for hyperthermic anaerobic cell apoptosis via a breast cup system in accordance with an embodiment of the present disclosure.

FIG. 3 is a heat transfer curve of a ratio of heat transfer through baseline anaerobic cell breast tissue in accordance with an embodiment of the present disclosure.

FIG. 4 is a heat transfer curve of a ratio of heat transfer through dead anaerobic cell breast tissue in accordance with an embodiment of the present disclosure.

FIG. 5 is a depiction of a prone breast cup system for hyperthermic anaerobic cell apoptosis in accordance with an embodiment of the present disclosure.

FIG. 6 is a depiction of a supine breast cup system for hyperthermic anaerobic cell apoptosis in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

Throughout the disclosure, the term “tourniquet-like suction (TSO)” defines a force similar to the force of gravity felt on a breast inside a cup in a face down or prone position. The pooling of blood that results from the prone position of the breast inside a breast cup in effect isolates vascular action in the breast cup from the rest of the body thereof. The TSO therefore to a higher degree vascularly isolates a breast from a patient's body to affect a substantial vascular circulation suspension in the breast to enable a hyperthermic stasis in the breast. Therefore, a radially overlapping and a radially sliding breast cup wall is configured for radially expanding for a prone position with respect to radially shrinking into itself for a supine position mechanically similar to a telescoping wall but in a radial configuration.

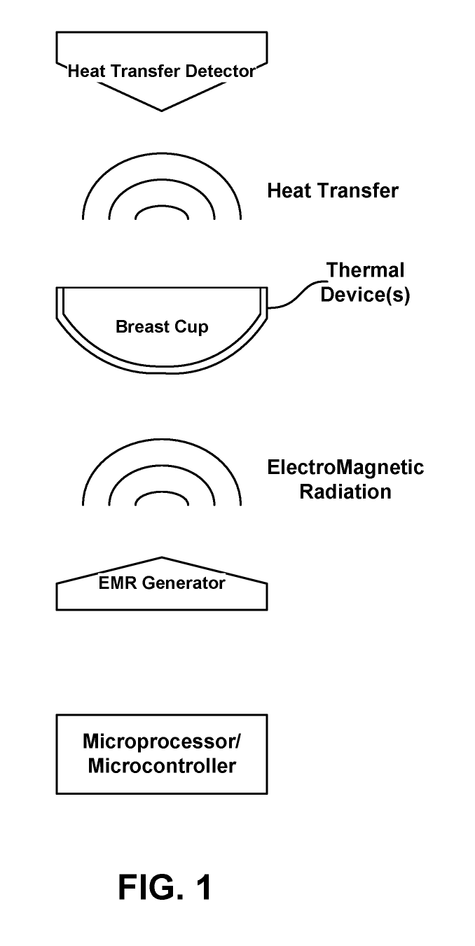

FIG. 1 is a component block diagram of a breast cup system for hyperthermic anaerobic cell apoptosis in accordance with an embodiment of the present disclosure. The components include a heat transfer detector, a breast cup comprising thermal device(s), an EMR (ElectroMagnetic Radiation) Generator and a Microcontroller/Microprocessor. Various thermal devices may be used separately or in combination to achieve anaerobic cell death. The generation of indigenous or latent heat within diseased cells via an ATP (adenosine triphosphate) response to electromagnetic radiation is also applied to the breast via the disclosed breast cup.

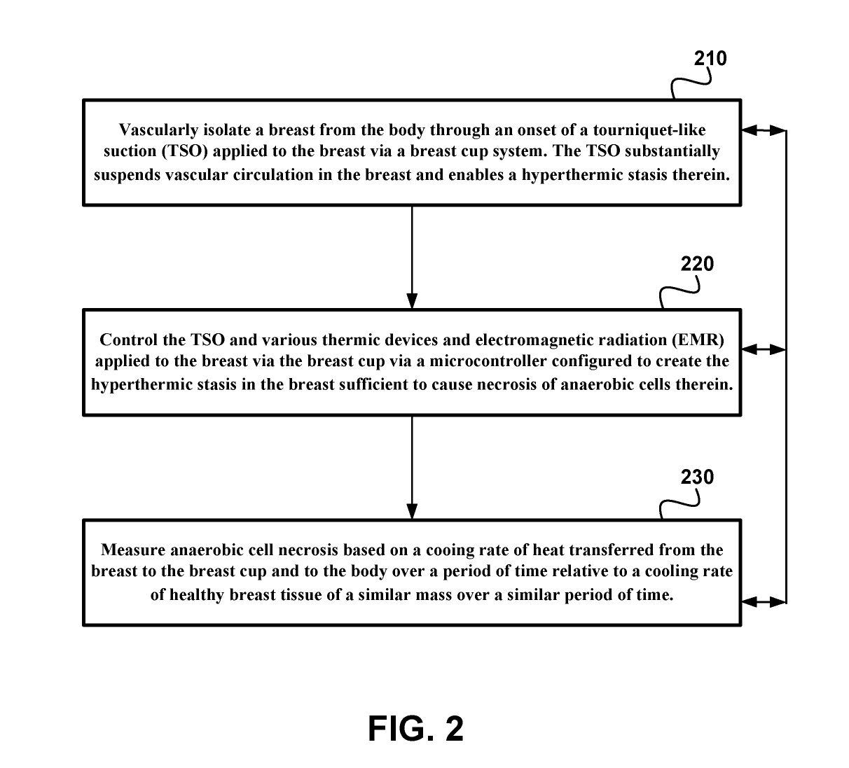

FIG. 2 is a block diagram of a method for hyperthermic anaerobic cell apoptosis via a breast cup system in accordance with an embodiment of the present disclosure. A method is also disclosed for anaerobic cell apoptosis via a breast cup system. The method includes 210 vascularly isolating a breast from the body thereof through an onset of a tourniquet-like suction (TSO) applied to the breast via a breast cup system. The TSO substantially suspends vascular circulation in the breast to enable a hyperthermic stasis in the breast. The disclosed method also includes 220 controlling the TSO and various thermic devices and electromagnetic radiation (EMR) applied to the breast via the breast cup and a microcontroller configured to create the hyperthermic isolation in the breast sufficient to cause apoptosis of anaerobic cells therein. The method further includes 230 determining the anaerobic cell apoptosis as a result of the TSO and the hyperthermic stasis based on one of an intrusive and a non-intrusive measurement including a ratio of an amount of heat transferred from the breast cup over a period of time through the breast relative to a heat transfer through a healthy breast tissue of a same mass over a same period of time.

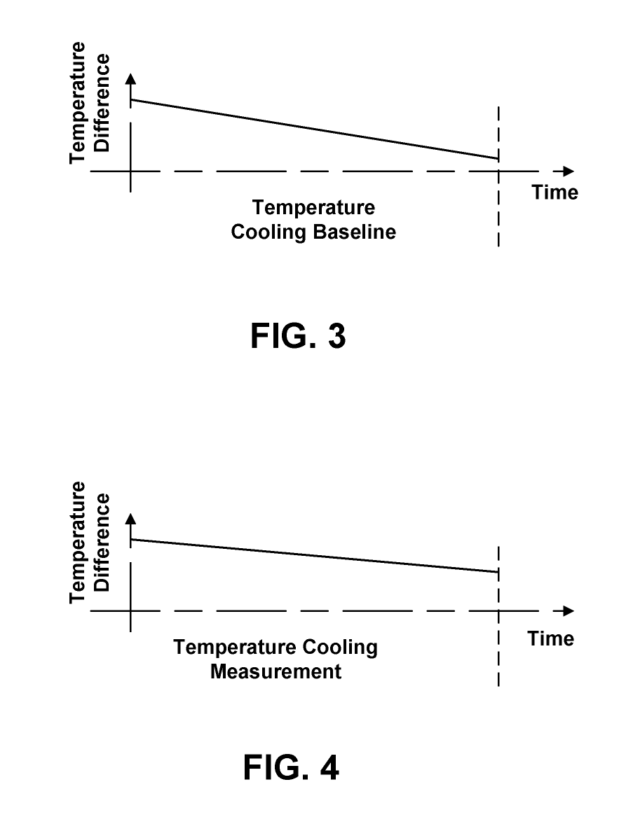

FIG. 3 is a heat transfer curve of a ratio of heat transfer through baseline anaerobic cell breast tissue in accordance with an embodiment of the present disclosure. Anaerobic breast mass is denser than surrounding healthy tissue. Therefore, heat transfer will be slower through the unhealthy tissue. Also, anaerobic cells are not able to dilate to dissipate heat and therefore slow down the heat transfer there through. These two factors both contribute to slower heat transfer through unhealthy breast tissue. Dead anaerobic cells are further constricted of vascular activity and therefore do not even have the benefit of heat transfer through blood vessels and further slow heat transfer through unhealthy tissue.

FIG. 4 is a heat transfer curve of a ratio of heat transfer through dead anaerobic cell breast tissue to the heat transfer through healthy breast tissue in accordance with an embodiment of the present disclosure.

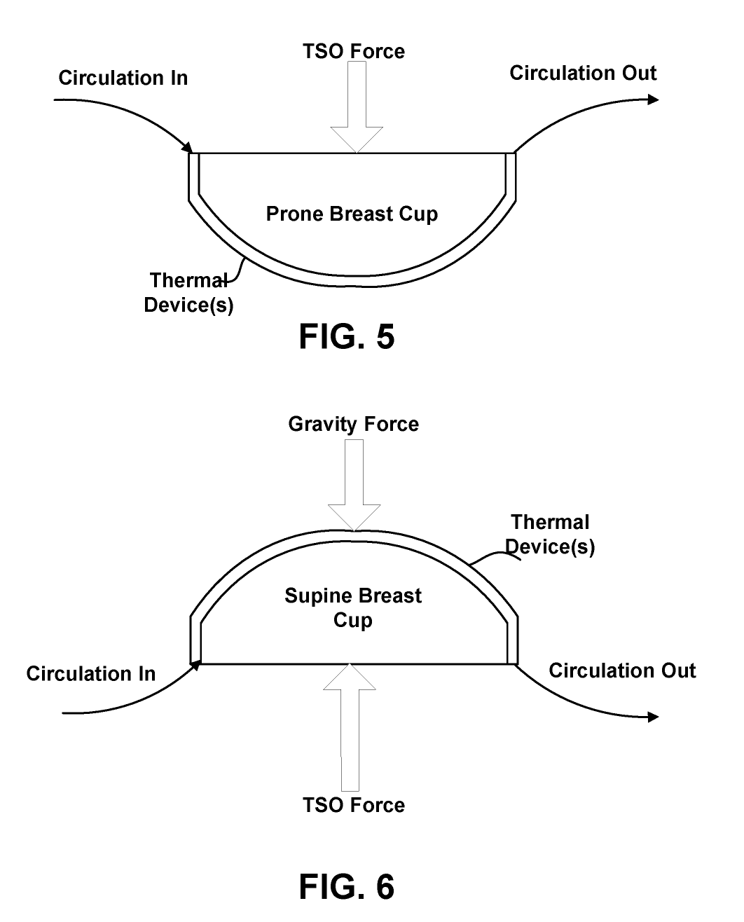

FIG. 5 is a depiction of a prone breast cup system for hyperthermic anaerobic cell apoptosis in accordance with an embodiment of the present disclosure. The TSO force needed in a prone breast cup system is less than the TSO force needed in a supine breast cup system due to gravity as an additive vector. Sizing the breast cup to the breast size has a marked value. As the breast is drawn into the breast cup through the TSO vacuum (using a slightly smaller cup than the breast) an event similar to crushing the capillary blood flow accrues inhibiting heat rejection.

FIG. 6 is a depiction of a supine breast cup system for hyperthermic anaerobic cell apoptosis in accordance with an embodiment of the present disclosure. Gravity works against the TSO force needed to set up the hyperthermic stasis for anaerobic cell apoptosis. The thermal devices may include one and more of the following either separately or in combination.

In an embodiment of the disclosure the various thermic devices include a heat transfer mechanism (resistive heat sheet) configured to surround the breast tissue drawn in the cup, the heat transfer mechanism configured to create the hyperthermic stasis in the breast tissue sufficient to cause apoptosis of anaerobic cells therein.

In another embodiment of the disclosure the various thermic devices include heated fluid(s) circulated over the breast tissue in the cup. The heated fluids are configured to create the hyperthermic stasis in the breast tissue sufficient to cause apoptosis of anaerobic cells therein.

In yet another embodiment of the disclosure the electromagnetic radiation (EMR) applied to the breast via the breast cup is configured to create a hyperthermic stasis in the breast tissue sufficient to cause apoptosis of anaerobic cells therein.

In a further embodiment of the disclosure, the hyperthermic isolation or stasis in the breast cup is maintained at or near an apoptosis threshold (106 degrees Fahrenheit plus or minus a ten percent tolerance) of anaerobic cells. This threshold varies on mass and density of the breast. Also, the breast is a human female breast but is not restricted to a human female breast. Additionally, the onset of the tourniquet-like suction (TSO) allows some blood flow to and from the breast but only at a steady state leakage amount to maintain healthy tissues but not to drop the hyperthermic stasis. The hyperthermic stasis is maintained for a length of approximately 40 minutes to an hour depending on a breast mass for the application of the TSO to the breast to prevent any permanent damage to healthy breast tissue. Furthermore, the suction created by the TSO is substantially similar to an upper range of an average healthy blood pressure of 80 to 120 mm Hg based on the blood pressure of a patient being treated by the breast cup system.

In a disclosed embodiment, a ratio of an amount of heat transferred from the breast cup over a period of time through the breast measured by a heat transfer detector relative to a heat transfer through a healthy breast tissue of a same mass over a same period of time greater than 1 determines anaerobic cell apoptosis. The ratio of heat transfer of healthy breast tissue to anaerobic cell apoptosis tissue varies over time and is similar at onset but over time becomes more biased toward faster heat transfer. Also, a heat transfer detector is used to determine the anaerobic cell apoptosis based on a cooing rate of heat transferred from the breast to the breast cup and to the body over a period of time relative to a cooling rate of healthy breast tissue of a similar mass over a similar period of time.

An embodiment of the method for anaerobic cell apoptosis via a breast cup system further includes determining a baseline heat transfer ratio and curve over time of the breast tissue before hyperthermia treatment and after hyperthermia treatment.

Embodiments of the disclosed method also include reducing the TSO force by a force equal to gravity when a patient is in a prone position as opposed to a supine position due to a pooling of blood in the breast. An intrusive measurement of the anaerobic cell apoptosis may include an anaerobic cell biopsy and other methods known in the art or yet unknown in the art of anaerobic cell diagnosis.

In other embodiments, a non-intrusive measurement of the anaerobic cell apoptosis includes an xray, a mammography, a tomography, a CAT (x-ray computerized axial tomography) scan, PET (positron emission tomography) scan, MRI (Magnetic Resonance Imaging) and other methods known in the art or yet unknown in the art of anaerobic cell diagnosis

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A breast cup system for improving breast health as well as anaerobic cell apoptosis:
a) a breast cup configured to vascularly isolate a breast from the body thereof via an onset of a tourniquet-like suction (TSO) applied to the breast, the TSO configured to substantially suspend vascular circulation in the breast to enable a hyperthermic stasis in the breast; and
b) a microcontroller configured to control the TSO and various thermic devices and electromagnetic radiation (EMR) applied to the breast via the breast cup to create the hyperthermic stasis in the breast sufficient to cause apoptosis of anaerobic cells therein;
c) a heat transfer detector configured to determine the anaerobic cell apoptosis based on a cooing rate of heat transferred from the breast to the breast cup and to the body over a period of time relative to a cooling rate of healthy breast tissue of a similar mass over a similar period of time.

2. The breast cup system of claim 1, wherein the various thermic devices include a resistive heat sheet configured to surround the breast tissue drawn in the cup, the heat sheet configured to create the hyperthermic stasis in the breast tissue sufficient to cause apoptosis of anaerobic cells therein.

3. The breast cup system of claim 1, wherein the various thermic devices includes heated fluid(s) circulated over the breast tissue in the cup, the heated fluids configured to create the hyperthermic stasis in the breast tissue sufficient to cause apoptosis of anaerobic cells therein.

4. The breast cup system of claim 1, wherein the electromagnetic radiation (EMR) applied to the breast via the breast cup is configured to create a hyperthermic stasis in the breast tissue sufficient to cause apoptosis of anaerobic cells therein.

5. The breast cup system of claim 1, wherein a hyperthermic isolation in the breast cup is maintained at or near a lowest 96.6 degrees Fahrenheit and a highest 106 degrees Fahrenheit plus or minus a ten percent tolerance.

6. The breast cup system of claim 1, wherein the breast is a human female breast.

7. The breast cup system of claim 1, wherein the onset of the tourniquet-like suction (TSO) allows some blood flow to and from the breast measured at only a steady state leakage amount.

8. The breast cup system of claim 1, further comprising a length of time, nominally 40 minutes to an hour depending on a breast mass sufficient for the application of the TSO to the breast for a desired level of blood cycling to prevent any permanent damage to healthy breast tissue.

9. The breast cup system of claim 1, wherein the suction created by the TSO is substantially similar to an upper range of an average healthy blood pressure of 80 to 120 mm Hg based on the blood pressure of a patient being treated by the breast cup system.

10. The breast cup system of claim 1, wherein on a ratio of an amount of heat transferred from the breast cup over a period of time through the breast measured by a heat transfer detector relative to a heat transfer through a healthy breast tissue of a same mass over a same period of time greater than 1 determines anaerobic cell apoptosis.

11. The breast cup system of claim 1, wherein the ratio of heat transfer of healthy breast tissue to anaerobic cell necrosis tissue varies over time and is similar at onset but over time becomes more biased toward faster heat transfer.

12. A method for anaerobic cell apoptosis via a breast cup system, the method comprising:
a) vascularly isolating a breast from the body thereof via an onset of a tourniquet-like suction (TSO) applied to the breast via a breast cup system, the TSO configured to substantially suspend vascular circulation in the breast to enable a hyperthermic stasis in the breast; and
b) controlling the TSO and various thermic devices and electromagnetic radiation (EMR) applied to the breast via the breast cup and a microcontroller configured to create the hyperthermic stasis in the breast sufficient to cause apoptosis of anaerobic cells therein;
c) determining the anaerobic cell apoptosis as a result of the TSO and the hyperthermic stasis based on one of an intrusive and a non-intrusive measurement including a ratio of an amount of heat transferred from the breast cup over a period of time through the breast relative to a heat transfer through a healthy breast tissue of a same mass over a same period of time.

13. The method for anaerobic cell apoptosis via a breast cup system of claim 12, further comprising determining a baseline heat transfer ratio and curve over time of the breast tissue before hyperthermia treatment and after hyperthermia treatment.

14. The method for anaerobic cell apoptosis via a breast cup system of claim 12, further comprising reducing the TSO force by a force equal to gravity when a patient is in a prone position as opposed to a supine position due to a pooling of blood in the breast.

15. The method of claim 14, further comprising a radially overlapping and a radially sliding breast cup wall configured for radially expanding for a prone position with respect to radially shrinking into itself for a supine position.

16. The method of claim 12, wherein a non-intrusive measurement of the anaerobic cell apoptosis includes an xray, a mammography, a tomography, a CAT (x-ray computerized axial tomography) scan, PET (positron emission tomography) scan, MRI (Magnetic Resonance Imaging) and other methods known in the art or yet unknown in the art of anaerobic cell diagnosis.

17. The method of claim 12, further including drawing a breast into a nominally smaller breast cup than a size of the breast based on a crushing of the capillary blood flow which accrues from an inhibiting heat rejection.

18. The method of claim 12, further including circulating hot air through conduits associated with the breast cup, the hot air adapted to contribute to the hyperthermic isolation of the breast and the onset of the TSO.

19. The method of claim 12, further including generating indigenous heat within diseased breast tissue from ATP generated by EMR applied to the breast and the breast cup.

20. The method of claim 12, wherein an intrusive measurement of the anaerobic cell apoptosis includes an anaerobic cell biopsy and other methods in the art of anaerobic cell diagnosis.
